(19) United States
(12) Patent Application Publication
(10) Pub. No.: US 2025/0197444 A1
(43) Pub. Date: Jun. 19, 2025

(54) Method of Preparing Procollagen from Freshwater Fish

(71) Applicant: NG Capital, LLC, Stowe, VT (US)

(72) Inventors: Fu Yu Hsieh, Taipei City (TW); Elizabeth Tien-Yang Cheng, Stowe, VT (US); Michael Furst, Stowe, VT (US)

(21) Appl. No.: 19/068,386

(22) Filed: Mar. 3, 2025

Related U.S. Application Data
(63) Continuation-in-part of application No. 18/135,116, filed on Apr. 14, 2023.

(51) Int. Cl. C07K 1/14

(52) U.S. Cl. CPC C07K 1/145

(57) ABSTRACT

A method of preparing procollagen from freshwater fish includes pressure treatment and separation of fish tissue, emulsification, homogenization, refrigeration, cleaning, extraction, homogenization, inactivation, homogenization, and filtration. In the mixing and emulsification step, freshwater fish tissue containing collagen is mixed with a surfactant to remove endotoxins to below 0.25 EU/ml. The procollagen is extracted following the step of adding enzymes and an acid solution of pH 3 to 6 to the pretreated tissue. The extraction technique can extract and retain greater amounts of procollagen while preserving the intact, tightly twisted triple-helix bonds of procollagen.

### RELATED APPLICATION DATA

This application is a continuation-in-part of U.S. patent application Ser. No. 18/135,116, filed Apr. 14, 2023, and titled “Method of Preparing Procollagen from Freshwater Fish,” which application is incorporated by reference herein in its entirety.

### FIELD OF DISCLOSURE

The present disclosure relates to the extraction of procollagen, particularly to a method of preparing procollagen from freshwater fish.

### BACKGROUND

Animal skin is comprised of 20% to 30% collagen extracellular matrix (ECM). Exposure to ultraviolet (UV) radiation and environmental pollution will cause loss of collagen in the skin, resulting in skin damage, roughness, and aging. In addition, many molecules such as the MMP enzyme family (matrix metallopeptidases or matrixins) can degrade the collagen ECM, making the skin structure unstable and causing skin aging.

Most of the collagen used in food, cosmetics, skincare, or pharmaceutical products on the market is in the form of collagen fragments. The loose, short-chain structure of collagen fragments makes them easily susceptible to decomposition by MMP enzymes. Moreover, due to their incomplete structure, collagen fragments cannot fully induce genetic responses of the collagen ECM. These products can only provide limited benefits for aging skin, because they are not particularly effective at activating the body's ability to repair aging skin.

Procollagen is a precursor of collagen characterized by a rod-like, central, triple-helical domain flanked by short linear telopeptides and globular N-terminal and C-terminal propeptides. FIG. 7 shows illustrations of procollagen, collagen, and a section of collagen fibril. As can be seen, the strand of procollagen 20 has a central triple-helical portion 25 with linear telopeptides 23a, 23b on each end with a globular N-terminal propeptide 22 and a globular N-terminal propeptide 24 (this particular structure may be referred to as type I procollagen, but for clarity will be referred to herein as procollagen). The strand of collagen 10 also has a central triple-helical portion 25 with linear telopeptides 23a, 23b on each end, but lacks the globular terminal propeptides. A section of collagen fibril 30 is comprised of a plurality of strands of collagen.

In organisms, procollagen synthesis is initiated within the lumen of the endoplasmic reticulum and continued within narrow enfolds of the plasma membrane. Procollagen molecules contain an uninterrupted ˜300 nm central Gly-X-Y region with about 1000 residues. This central region is flanked by non-helical (non Gly-X-Y) N- and C-terminal propeptide ends.

In this way, the procollagen molecule comprises three polypeptide α-chains supercoiled into a right-handed helix of about ˜300 nm, where each α-chain is a left-handed polyproline-II helix with a central Gly-X-Y repeat region. On each side of this triple helical region are non-helical N- and C-terminal ends.

Cleavage of the non-helical sequences produces a triple helical unit of collagen, tropocollagen that includes short non-helical ends of telopeptides. The tropocollagen is used to form collagen. Because procollagen has a different structure than collagen, it has different properties, such as denaturation temperature, and interacts with reagents differently, than procollagen. Procollagen has a higher denaturization temperature than collagen. The reason procollagen has a high denaturation temperature is due to the complete structure with the intact N-terminus and C-terminus, which collagen does not have. This structure is stable and strong can be very difficult to break.

Procollagen has been found to effectively protect and repair skin tissues damaged by UV radiation. In the skin, collagen fibrils are formed from the tightly twisted triple helical structure of procollagen, which promote the proliferation of fibroblasts, and induce the secretion of growth factors to form the collagen ECM, protecting the skin from light damage.

The tightness and amount of procollagen are closely related to the age of the skin. The collagen fibrils constructed by procollagen in mature skin are looser and shorter in chain length, resulting in a decrease in the number of fibroblasts and instability of the collagen ECM. Additionally, loose collagen fibrils are easily broken down into collagen fragments by MMP enzymes, leading to a reduced or lost ability to protect the skin. On the other hand, collagen in the human body can attract fibroblasts to attach and proliferate, and promote the formation of ECM, thereby providing skin protection. Moreover, the structure of collagen can induce and actively initiate the UV signaling pathway for anti-UV repair.

The tightly twisted triple helical structure of procollagen makes it less susceptible to external environmental damage and more suitable than collagen fragments for use in skincare, medical-grade cosmetic, and medical products. Currently, procollagen can be synthesized but cannot be effectively extracted from animal tissues, making it a difficult material to produce in large quantities.

Further, contamination from endotoxins is common in collagen containing structures. This is caused by gram-negative bacteria, which are commonly found in animals' habitats and can adhere to the skin or be absorbed into the body of animals, and during the process of extracting procollagen from animal tissue, these bacteria will die and release lipopolysaccharides (also known as endotoxins). Endotoxins can be dangerous when they enter the bloodstream, causing microcirculation disorders, septic shock, disseminated intravascular coagulation, and fever. In addition, the human body is extremely sensitive to the pyrogenic effects of endotoxins. Even tiny amounts (1-5 ng/kg body weight) of endotoxins injected into the body can cause fever and other harm.

Endotoxins are non-protein compounds with stable structures that must be heated at 250° C. for 2 to 4 hours to destroy their activity. Unfortunately, currently available collagen on the market has a denaturation temperature below 100° C., which makes it difficult to remove endotoxins using via heating. Additionally, soaking collagen in alcohol and acetone can remove endotoxins, but this method often results in protein denaturation. Some literature suggests that endotoxins can be removed by using Triton X-114 cloud point extraction, but this method can reduce the endotoxins only from 10 million EU/mL to around 100,000 EU/mL, which is still well above the allowable endotoxin limits for clinical applications and may leave residual Triton X-114 as well. Currently, lowering endotoxin levels to below 0.25 EU/mL requires a special column chromatography technique, which is very expensive and cannot be used for industrial production.

Current industry practices for effectively removing endotoxins from collagen materials to meet medical standards often involve pretreatment with sodium hydroxide (NaOH). However, the drawback is that NaOH can destroy protein structures, causing denaturation and the inability to achieve optimal protein functionality. In addition, NaOH can also reduce the yield of protein extraction.

Specifically, the processing time and concentration of NaOH used in tissue treatment will affect the tightness, length, and thus denaturation temperature of collagen fibrils. In addition, in the case of industrial production, the excessive use of NaOH solutions will generate a large amount of waste liquid, causing environmental pollution.

Soaking collagen materials in alcohol and acetone can remove endotoxins, but this method often results in protein denaturation.

Currently, for production scales, procollagen can only be synthesized; it cannot be effectively extracted from animal tissues.

Therefore, there is a need for effective, efficient methods to extract procollagen from animal tissues while reducing endotoxins and melanin to medically approved levels and increasing the denaturation temperature of the extracted procollagen.

### SUMMARY OF DISCLOSURE

A method is provided for extracting tight twisted triple helix procollagen from freshwater fish. The extraction includes a sequence of pressure extrusion, mechanical reduction, mixing, and emulsification followed by homogenization, refrigeration and cleaning. Then an enzymatic extraction process is used followed by multiple rounds of homogenization along with inactivation and finally, filtration.

The result is a low cost, high volume method of producing procollagen with allowably low endotoxin levels (below required limits for clinical applications).

Neither NaOH nor alcohol are used in the disclosed process and furthermore, the process produces high yields of procollagen (up to 80%) compared to processes that use NaOH (yields of 3% or significantly less).

In an embodiment, a method of preparing procollagen from freshwater fish includes the following steps:
i. Pressure treatment: Using high-pressure extrusion to remove blood, water, and fat from freshwater fish tissue;
ii. Mechanically breaking down the freshwater fish tissue by crushing and/or cutting or by use of air blowing the extruded the freshwater fish tissue into chunks, strips, or a powder;
iii. Mixing the freshwater fish tissue particles with a surfactant (preferably a nonionic surfactant), a salt electrolyte, and urea to obtain an emulsion solution;
iv. Homogenizing the emulsion solution for 13 to 20 minutes;
V. Refrigerating the emulsion solution at 0° C. to 10° C. for 5 to 60 minutes;
vi. Washing the emulsion solution with water at 38° C. to 42° C. until the absorbance value of the cleaning solution at 200 nm to 300 nm approaches zero (to reduce endotoxins in the freshwater fish tissue to below 0.25 EU/mL);
vii. Mixing the freshwater fish tissue with an enzyme and an acid solution of pH 3 to 6 to obtain an extract solution;
viii. Homogenizing the extract solution at 4° C. to 75° C. for 2 to 48 hours;
ix. Adding an enzyme inhibitor to the extract solution to lower the pH to below 3;
X. Homogenizing the inactivated extract solution at 4° C. to 75° C. for 2 to 48 hours; and
xi. Separating the supernatant from the homogenized extract solution through filtration, centrifugation, and/or sieving to obtain procollagen.

In some embodiments, the salt is NaCl and is added in the amount of between 1 to 5 wt %.

In some embodiments, the urea is added in an amount of between 3 to 10 wt %.

In some embodiments, the surfactant is selected from the group consisting of Tween 20, Tween 80, Triton X-100, and mixtures thereof, and added in the amount of between 0.05 and 0.5 wt %.

In some embodiments, the enzyme activity of enzyme is between 20 and 2000 U, and said enzyme is added in the amount of between 0.5 and 10 wt %.

In some embodiments, the enzyme is selected from the group consisting of papain, bromelain, and mixtures thereof.

In some embodiments, the acid solution is selected from the group consisting of acetic acid, citric acid, lactic acid, and mixtures thereof, and added in the amount of between 0.5 to 1M.

In some embodiments, the enzyme mixtures are a combination of bromelain and papain, and the mixing ratio of bromelain to papain is between 0:1 and 1:2.

In some embodiments, the enzyme inhibitor is selected from acetic acid, lactic acid and mixtures thereof, and the mixing ratio of acetic acid to lactic acid is between 1:0 and 1:3.

In some embodiments, the electrolyte is a salt having the form Na—X in which X is a halide ion.

In some embodiments, the mechanical separation of the freshwater fish tissues further comprises cutting and crushing (or using air blowing) the freshwater fish tissues into one member selected from the group consisting of: powder, chunks, strips and combinations thereof.

In some embodiments, the separation of the supernatant from the homogenized extract solution further comprises one member selected from the group consisting of filtration, centrifuging, sieving and combinations thereof.

### BRIEF DESCRIPTION OF THE DRAWINGS

FIG. 1 is a table summarizing the steps for procollagen preparation in accordance with an embodiment of the present disclosure.

FIGS. 2A-2C are photos of freshwater fish particles before and after the removal of melanin and endotoxins in the embodiments and comparative examples.

FIGS. 2D-2G are photos of the products (supernatant and sediments) of the embodiments and comparative examples following the extraction of procollagen.

FIG. 3A is a Fourier Transform Infrared (FTIR) spectrogram of Embodiment 1.

FIG. 3B is a FTIR spectrogram of Comparative Example 1.

FIG. 4A is a dynamic light scattering (DLS) spectrogram of Embodiment 1.

FIG. 4B is a DLS spectrogram of Comparative Example 1.

FIG. 5A is an electropherogram of a sodium dodecyl sulfate-polyacrylamide gel electrophoresis (SDS-PAGE) of all the embodiments disclosed herein.

FIG. 5B is an SDS-PAGE electropherogram of Comparative Examples #1 and #2.

FIG. 6A is a graph of the differential scanning calorimetry (DSC) of Embodiment 1.

FIG. 6B is a graph of the DSC of Comparative Example 1.

FIG. 7 (prior art) includes illustrations depicting the structures of procollagen, collagen, and collagen fibril.

FIG. 8 is a table summarizing the amino acid composition analysis of Embodiment 1.

### DETAILED DESCRIPTION

Procollagen, when extracted from freshwater fish tissue may be used to promote the generation and stability of extracellular matrix in the human body, thereby protecting the skin. A process is provided for extracting procollagen from freshwater fish tissue that is suitable for medical and commercial use.

The process effectively removes endotoxins and melanin found in freshwater fish tissue to prepare structurally intact procollagen (i.e., procollagen that has not broken down into collagen or collagen peptides) that is stable and meets the standards for medical use without using NaOH.

The process of preparing procollagen from freshwater fish includes the steps of:
Using high-pressure extrusion to remove blood, water, and fat from freshwater fish tissue containing collagen without the use of NaOH;
Air blowing, or cutting and/or crushing, the extruded freshwater fish tissue into fish tissue particles such as chunks, strips, or a powder, or doing so via air blowing;
Mixing the freshwater fish tissue particles with a surfactant (preferably a nonionic surfactant), a salt (e.g., Na—X such as NaCl), and urea to obtain an emulsion solution;
Homogenizing the emulsion solution for 13 to 20 minutes;
Refrigerating the homogenized emulsion solution at 0° C. to 10° C. for 5 to 60 minutes;
Washing the emulsion solution with water at 38° C. to 42° C. until the absorbance value of the cleaning solution at wavelengths in the range of 200 nm to 300 nm approaches zero to reduce the level of endotoxins below 0.25 EU/ml;
Mixing the washed emulsion with an enzyme and an acid solution of pH 3 to 6 to obtain an extract solution;
Homogenizing the extract solution at 4° C. to 75° C. for 2 to 48 hours;
Adding an enzyme inhibitor to the extract solution to lower the pH to below 3;
Homogenizing the inactivated extract solution at 4° C. to 75° C. for 2 to 48 hours; and
Separating the supernatant from the homogenized extract solution through filtration, centrifugation, and/or sieving to obtain procollagen.

In an embodiment, NaCl is added in the amount of between 1 and 5 wt %.

In an embodiment, the urea is added in the amount of between 3 and 10 wt %.

In an embodiment, the surfactant is selected from one of the group consisting of Tween 20, Tween 80 and Triton X-100, and mixtures thereof, and added in the amount of between 0.05 and 0.5 wt %.

In an embodiment, the enzymatic activity of enzyme can range from 20 to 2000 U, and the enzyme is added in the amount of between 0.5 and 10 wt %.

In an embodiment, enzyme is selected from papain or bromelain or mixtures thereof.

In an embodiment, the acid solution is selected from the group consisting of acetic acid, citric acid, lactic acid and mixtures thereof, and added in the amount of between 0.5 to 1M.

In an embodiment, the enzyme mixture is a combination of bromelain and papain, and the mixing ratio of bromelain to papain ranges from 0:1 to 1:2.

In an embodiment, the enzyme-inhibitor is selected from acetic acid, lactic acid, and mixtures thereof, and the mixing ratio of acetic acid to lactic acid ranges from 1:0 to 1:3.

In this way, procollagen from freshwater fish is obtained without using NaOH cleaning for animal tissues in raw materials. The process also effectively removes pigments and endotoxins to below 0.25 EU/ml, making the extracted procollagen clinically applicable by medical standards.

Furthermore, the extraction time is less than existing extraction techniques and yields of 60% to 80% procollagen can be obtained. The preparation method of procollagen can increase the denaturation temperature of the processed procollagen and prepares procollagen that may be safe for use in medical-grade cosmetics, skincare, and medical purposes.

In addition, the procollagen extracted as described herein retains structurally intact and tightly twisted triple helices.

### Implementation, and Test Results Compared to Other Methods

The following are specific embodiments illustrating methods of preparing collagen from freshwater fish.

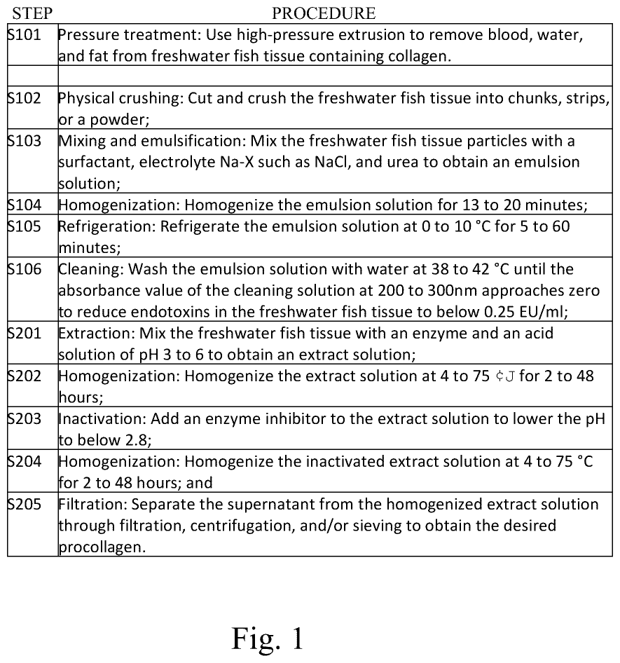

As outlined in FIG. 1, a method of extracting procollagen from fish includes the following steps: S101 pressure treatment, S102 breaking down the tissue into smaller particles, S103 mixing and emulsification, S104 homogenization, S105 refrigeration, S106 cleaning, S201 extraction, S202 homogenization, S203 inactivation, S204 homogenization, and S205 filtration.

Specifically, steps S101 through S106 are the process of pretreatment used to remove pigments and endotoxins from freshwater fish tissue. Steps S201 through S205 are the process of extraction used to obtain procollagen from the treated freshwater fish tissue. These steps are preferably performed in the order discussed below.

S101 Pressure treatment: high-pressure extrusion is used to remove blood, water, and fat from freshwater fish tissue. The freshwater fish tissue can be fish skin and/or fish organ tissue. In a preferred embodiment, this step does not involve the use of NaOH.

S102 Breaking down: the extruded freshwater fish tissue is broken down into smaller particles, such as chunks, strips, or powder, or, preferably, this step is accomplished via air blowing to avoid applying shear forces to the fish tissue.

S103 Mixing and emulsification: the freshwater fish tissue particles are mixed with a surfactant, which is preferably a nonionic surfactant, a salt, such as NaCl, and urea, to obtain an emulsion solution.

Preferably, the surfactant is a solution selected from Tween 20, Tween 80, and Triton X-100, although combinations thereof may be employed. The surfactant is added in the amount of between 0.05 and 0.5 wt %. The NaCl (or other sodium halide salt) is added can be used in the amount of between 1 to 5 wt %. The urea is added in the amount of between 3 to 10 wt %.

NaCl increases the osmotic pressure in the extracellular fluid, which can cause a pressure difference between the extracellular fluid and intracellular fluid, thereby inhibiting the release of endotoxins by bacteria. This is because high osmotic pressure in the extracellular fluid causes loss of intracellular water, leading to cell shrinkage, thus reducing bacteria cell death and the release of endotoxins.

In addition, NaCl can further reduce the release of endotoxins by inhibiting the breakdown of the cell wall and the release of proteases.

S104 Homogenization: Homogenize the emulsion solution using ultrasound, stirring, and shaking methods for 13 to 20 minutes.

S105 Refrigeration: Refrigerate the emulsion solution at 0° C. to 10° C. for 5 to 60 minutes. Preferably, the refrigeration temperature is 4° C.

S106 Cleaning: Wash the emulsion solution with water at 38° C. to 42° C. until the absorbance value of the emulsion solution at a selected wavelength in the range of 200 nm to 300 nm approaches zero to reduce the endotoxins to below 0.25 EU/ml in the emulsion solution. As used herein, “approaches zero” means an absorbance value of less than about 0.1, where “about” means+/−10% or +/−5%.

S201 Extraction: Mix the washed emulsion solution with an endotoxin level of below 0.25 EU/ml with an enzyme and an acidic solution of pH 3 to 6 to obtain an extract solution.

Specifically, the enzymatic activity of the enzyme can range from 20 to 2000 U, preferably 2000 U. Furthermore, the enzyme may be selected from the group consisting of papain, bromelain, and mixtures thereof, and added in the amount of between 0.5 to 10 wt %. The acid solution may be selected from the group consisting of acetic acid, citric acid, lactic acid, and mixtures thereof, and added in the amount of between 0.5 to 1M.

Preferably, the enzyme solution is a mixture of bromelain and papain. The mixing ratio of bromelain to papain ranges from 0:1 to 1:2.

S202 Homogenization: Homogenize the extract solution using ultrasound, stirring, and/or shaking at 4° C. to 75° C. for 2 to 48 hours.

S203 Inactivation: Lower the pH of the extract solution to below pH 3 using an enzyme inhibitor, preferably below pH 2.8.

The enzyme inhibitor is selected from acetic acid and lactic acid, preferably a mixture of both. The mixing ratio of acetic acid to lactic acid ranges from 1:0 to 1:3.

S204 Homogenization: Homogenize the inactivated extract solution using ultrasound, stirring, and/or shaking at 4° C. to 75° C. for 2 to 48 hours.

S205 Filtration: Collect the supernatant through filtration, centrifugation, and/or sieving of the extract solution to obtain the desired procollagen.

### EMBODIMENTS AND COMPARATIVE EXAMPLES

Three embodiments are described that use the concentrations and ratios provided below for the seps described above. For comparison purposes, extractions were also performed using current methods (described in Comparative Examples 1-2 below).

### Embodiment 1

A summary reagents, their amounts, and extraction time for Embodiment 1 is shown in Table 1.

TABLE 1
Embodiment 1

| | Surfactant | NaCl | Urea | Acid | Enzyme | Extraction |
|---|---|---|---|---|---|---|
| NaOH | (wt %) | (wt %) | (wt %) | (M) | (wt %) | time (hr) |
| — | Tween 80: | 1 wt % | 5 wt % | Acetic acid:lactic | bromelain:papain | 8 hrs |
| | 0.05 wt % | | | acid 1:1.5 | 1:2 | |
| | | | | Adjust the pH of the | | |
| | | | | acid solution to 4 | | |
| | | | | for extraction | | |

### Embodiment 2

A summary reagents, their amounts, and extraction time for Embodiment 2 is shown in Table 2.

TABLE 2
Embodiment 2

| | Surfactant | NaCl | Urea | Acid | Enzyme | Extraction |
|---|---|---|---|---|---|---|
| NaOH | (wt %) | (wt %) | (wt %) | (M) | (wt %) | time (hr) |
| — | — | 5 wt % | 10 wt % | Acetic acid:lactic | bromelain:papain | 8 hrs |
| | | | | acid 1:1 | 1:1 | |
| | | | | Adjust the pH of the | | |
| | | | | acid solution to 4 | | |
| | | | | for extraction | | |

### Embodiment 3

A summary reagents, their amounts, and extraction time for Embodiment 3 is shown in Table 3

TABLE 3
Embodiment 3

| | Surfactant | NaCl | Urea | Acid | Enzyme | Extraction |
|---|---|---|---|---|---|---|
| NaOH | (wt %) | (wt %) | (wt %) | (M) | (wt %) | time (hr) |
| — | Tween 80 | 1 wt % | 3 wt % | Acetic acid; | bromelain:papain | 8 hrs |
| | 0.05 wt % | | | Adjust the pH of the | 0:1 | |
| | | | | acid solution to 4 | | |
| | | | | for extraction | | |

### COMPARATIVE EXAMPLES

A fragment of freshwater fish tissue was prepared and subjected to high-pressure extrusion to remove blood, water, and fat. The fragment was then broken into chunks, strips, or a powder through cutting and crushing. Next, the particles of the freshwater fish tissue were washed with a NaOH solution until the endotoxins in the freshwater fish tissue were reduced to below 0.25 EU/ml.

In Comparative Example 1, the particles of freshwater fish tissue were cleaned with NaOH at 4° C. for 24 hours; in Comparative Example 2, the particles of freshwater fish tissue were cleaned with NaOH at 4° C. for 30 hours.

The particles cleaned with NaOH were then mixed with 0.8 M acetic acid and 0.1 wt % porcine pepsin to obtain an extract solution. The extract solution was homogenized at 4° C. and then extracted for 240 hours. The supernatant that was formed following the extraction was then collected through filtration, centrifugation, and/or sieving to obtain procollagen.

### Comparative Example 1

The formula composition of comparative example 1 is as shown in Table 4.

TABLE 4
Comparative Example 1

| | Surfactant | NaCl | Urea | Acid | Enzyme | Extraction |
|---|---|---|---|---|---|---|
| NaOH | (wt %) | (wt %) | (wt %) | (M) | (wt %) | time (hr) |
| 0.1M | — | — | — | Acetic | Porcine | 240 hrs |
| | | | | acid; | pepsin | |
| | | | | 0.8M | 0.1 wt % | |

### Comparative Example 2

Comparative Example 2 was conducted using the implementation steps described in Comparative Example 1, and its formulation was adjusted as shown in Table 5.

TABLE 5
Comparative Example 2

| | Surfactant | NaCl | Urea | Acid | Enzyme | Extraction |
|---|---|---|---|---|---|---|
| NaOH | (wt %) | (wt %) | (wt %) | (M) | (wt %) | time (hr) |
| 0.5M | — | — | — | Acetic | Porcine | 240 hrs |
| | | | | acid; | pepsin | |
| | | | | 0.8M | 0.1 wt % | |

### Observation of Pigment Removal Effect

Pretreatments of freshwater fish tissue fragments for endotoxin removal were conducted in all of the embodiments and comparative examples, and observation and a comparison of an untreated fragment and the pretreated fragments were performed with the naked eye. The untreated fragment and the pretreated fragments from the comparative examples appeared light grey, whereas the fragments from the embodiments clearly appeared off-white, indicating that the embodiments likely removed more melanin than the comparative examples.

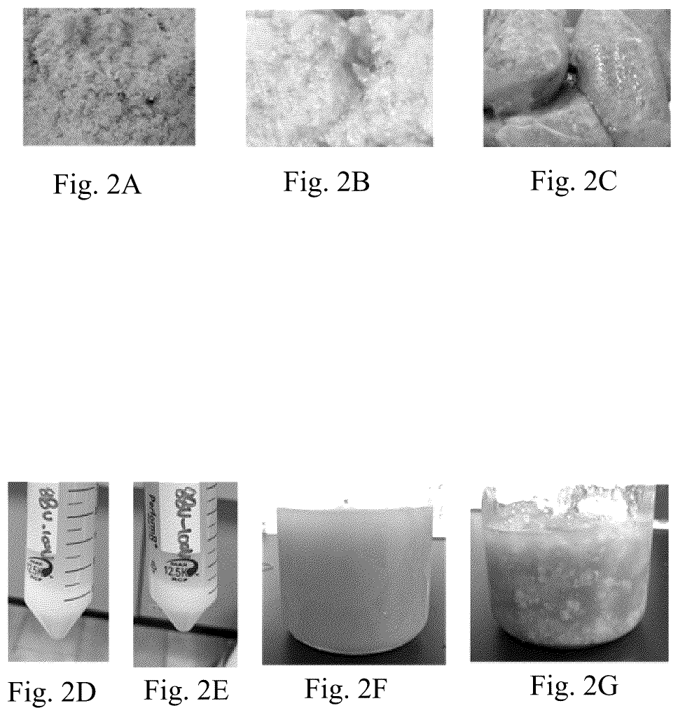

FIGS. 2A-2C are photos showing:
2A is of organ particles of freshwater fish containing before pretreatment;
2B is of organ particles of freshwater fish containing after being pretreated with a surfactant, urea, and NaCl, and then washed with clean water in accordance with embodiments of the present disclosure; and
2C is of particles of freshwater fish containing after being pretreated with NaOH in accordance with previous techniques.

FIG. 2D is a photo of a tube containing the material shown in FIG. 2B after the extraction process with papain and acetic acid;
FIG. 2E is a photo of a tube containing the material shown in FIG. 2B after the extraction process with a mixture of bromelain and papain, then a mixture of lactic acid and acetic acid, and then centrifuged, to get the supernatant and sediments; and
FIGS. 2F and 2G are photos of a beaker containing freshwater fish skin powder after being pretreated with NaOH and then mixed with acetic acid and porcine pepsin, to get supernatant (2F) and sediments (2G).

### Fourier Transform Infrared (FTIR) Spectroscopy

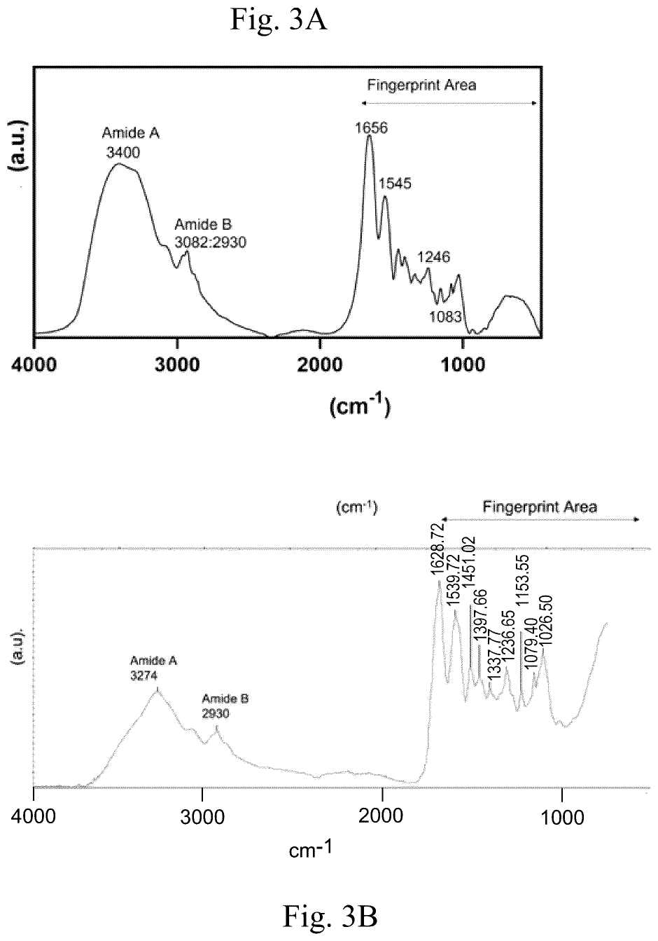

FIG. 3A is the Fourier transform infrared FTIR spectrogram of procollagen extracted in accordance with embodiments of this disclosure using a mixture of papain and bromelain and a mixture of acetic acid and lactic acid.

As can be seen in FIG. 3A, FTIR spectroscopy can distinguish several characteristic absorption bands of collagen: Amide I at around 1656 cm⁻¹, Amide II at around 1545 cm⁻¹, and a set of three weaker bands representing the Amide III vibration mode centered at around 1246 cm⁻¹.

The Amide I band arises from the stretching and bending vibrations of the peptide carbonyl group (—CO). A peak at 1083 cm⁻¹ appears in the spectrum of fibrous procollagen and is slightly shifted from the peaks of Amide I and Amide II. Therefore, the spectra in FIG. 3A, including the specific feature appearing as a peak at 1083 cm⁻¹, allows for the confirmation of the presence of procollagen.

FIG. 3B is a FTIR spectrogram of procollagen obtained from an extraction done according to the process for the Comparative Examples noted above. It can be seen from the spectra that there is a peak at 3274 cm⁻¹, which is Amide A. It is found that compared to the spectra in FIG. 3A, there is a shift in the peak position, and the peak height of Amide A is lower than that in FIG. 3A, indicating that the alpha helix structure in the procollagen obtained from the Comparative Example process is less stable and has a lower content than the procollagen obtained through the process described for the embodiments. The same situation is observed for Amide B. In addition, there is a small peak at 1083 cm⁻¹, which suggests the presence of only a small amount of procollagen.

### Triple Helix Characteristic Test

The ultraviolet-visible (UV-VIS) absorption spectrum of a protein is mainly determined by the peptide bonds or side chains of the protein. The amino acid sequence of the protein contains glycine, proline, and hydroxyproline, and the triple-helix procollagen has a maximum peak at about 240 nm.

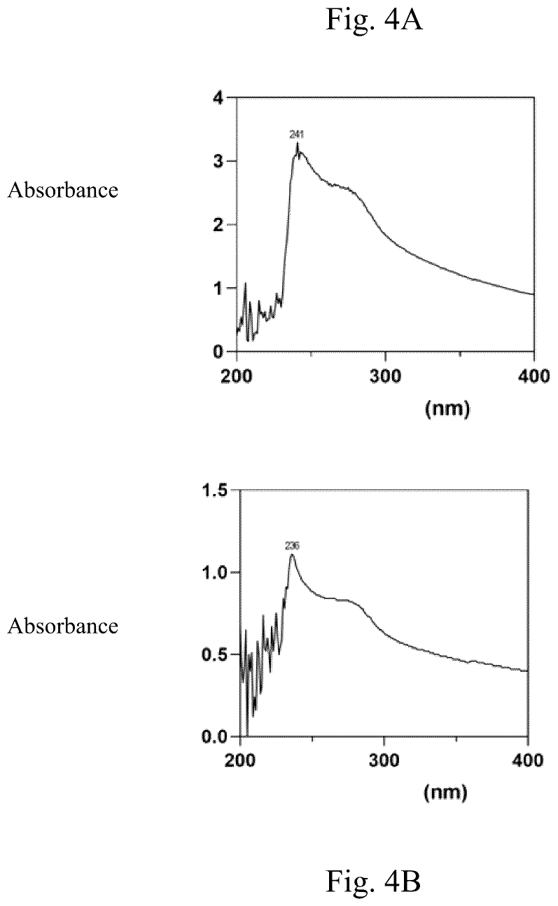

FIG. 4A is the dynamic light scattering (DLS) spectrogram of procollagen extracted according to this disclosure after an extraction with a mixture of bromelain and papain and a mixture of lactic acid and acetic acid. As can be seen in FIG. 4A, DLS of Embodiment 1 showed a maximum absorbance at around 240 nm, with no substantial peaks at other wavelengths, indicating that the triple helix of Embodiment 1 is tightly twisted, and no collagen fragments were present, demonstrating that Embodiment 1 included intact procollagen.

FIG. 4B is the DLS spectrogram of procollagen obtained through the process described for the Comparative Examples. As can be seen in FIG. 4B, the spectrum of procollagen obtained by Comparative Example 1 showed a maximum absorbance at around 240 nm, but the absorbance was not as high as that of Embodiment 1, indicating that the amount of triple helical procollagen in Comparative Example 1 was lower than that in Embodiment 1. Moreover, Comparative Example 1 also showed more substantial absorbance peak signals between 200 and 240 nm, indicating that Comparative Example 1 included collagen fragments, which absorb at these wavelengths. In other words, compared to Embodiment 1, the purity of procollagen obtained from Comparative Example 1 was lower.

### Sodium Dodecyl Sulfate Polyacrylamide Gel Electrophoresis

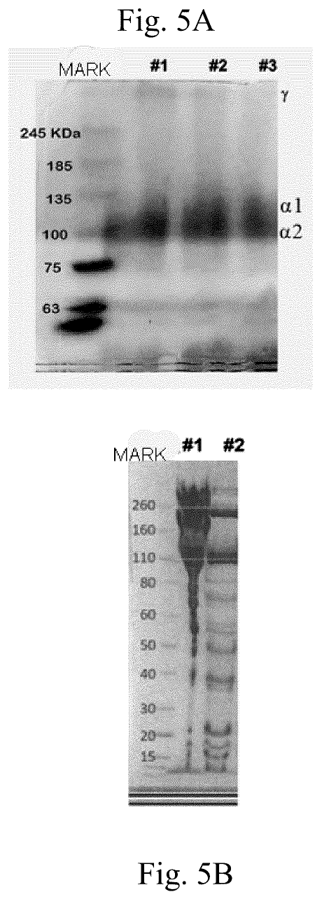

FIG. 5A shows the electropherogram of sodium dodecyl sulfate polyacrylamide gel electrophoresis (SDS-PAGE) of procollagen of Embodiment 1 (labeled #1), Embodiment 2 (labeled #2), and Embodiment 3 (labeled #3).

As can be seen in FIG. 5A, the result of SDS-PAGE shows that Embodiment 1 has α1 and α2 chains, indicating that it is procollagen. In addition, the γ-chain (trimer) also shows the presence of high molecular weight cross-linking.

FIG. 5B shows the SDS-PAGE electropherogram of:
(#1) material obtained from freshwater fish tissue of Comparative Example 1 after a pretreatment with NaOH for 24 hours and then an extraction with a mixture of acetic acid and porcine pepsin for 240 hours; and
(#2) material obtained from freshwater fish tissue of Comparative Example 1 after a pretreatment with NaOH for 30 hours and then an extraction with a mixture of acetic acid and porcine pepsin for 240 hours.

As shown in FIG. 5B, for both Comparative Example 1 treated with NaOH for 24 hours and Comparative Example 2 treated for 30 hours, not only were α1 and α2 chains and γ chains present but so was a low molecular weight sequence fragment. Compared to Embodiment 1, there were many low molecular weight sequence fragments in the collagen of Comparative Examples 1 and 2, indicating that the triple-helix structure of procollagen in Comparative Example 1 was not able to maintain its characteristics and was easily degraded into small collagen fragments.

### Cell Cytotoxicity Test

The cell cytotoxicity test was conducted on mouse fibroblast cells (L929) in vitro to evaluate the effect of Embodiment 1 on the survival rate of L929. Cell survival rate was assessed using MTT assay analysis. According to the ISO10993-1 method for cell cytotoxicity, after treatments for 24, 48, and 72 hours, the survival rates were approximately 75%, 82%, and 88%, respectively, indicating that the procollagen obtained from Embodiment 1 does not exhibit cell toxicity.

### Denaturation Temperature

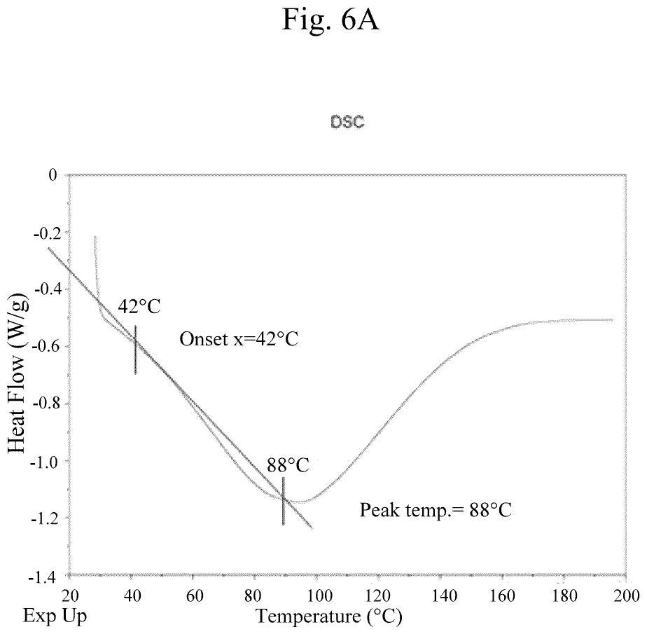

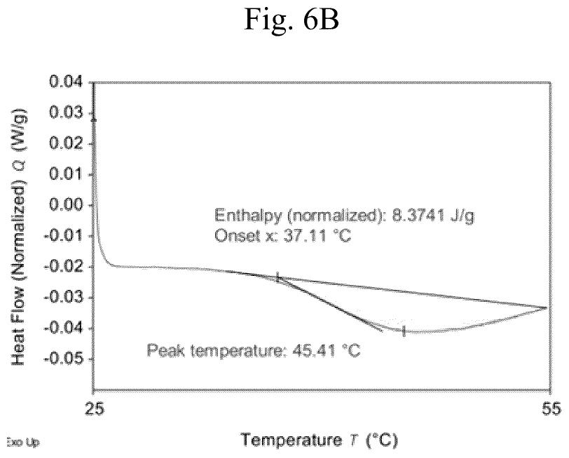

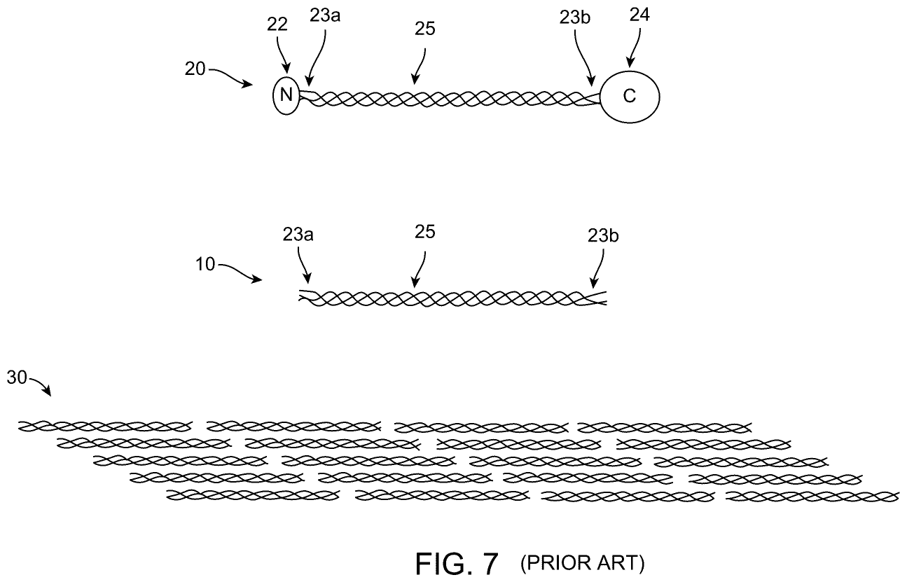

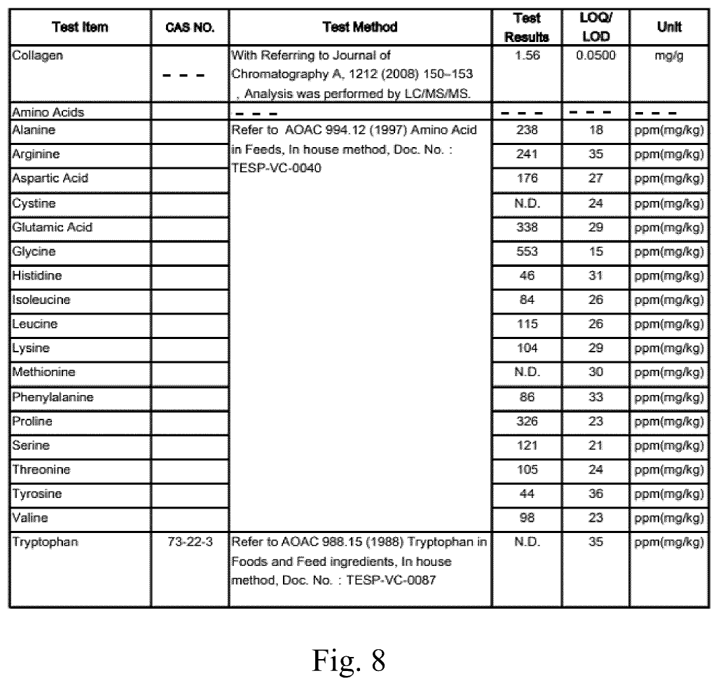

The denaturation temperature was determined using differential scanning calorimetry (DSC), and the test results are recorded in Table 6 and FIGS. 6A and 6B. FIG. 6A is the graph of DSC of procollagen extracted according to embodiments described herein after an extraction with a mixture of papain and bromelain and a mixture of acetic acid and lactic acid. FIG. 6B is a graph of DSC of procollagen extracted according to the Comparative Examples.

As shown in FIG. 6A, the denaturation temperatures of procollagen obtained via the embodiments were approximately between 77° C. and 88° C., whereas the denaturation temperatures of procollagen obtained from the comparative examples (see FIG. 6B) were approximately 45° C. This suggest that procollagen extracted by Embodiment 1 has a more tightly packed/twisted triple-helix structure than procollagen extracted by the Comparative Examples.

### Protein Analysis

The protein concentration and amino acid sequence were analyzed using LC-MS/MS. FIG. 9 shows the analysis of the amino acid composition of procollagen obtained using the process of the embodiments after an extraction with a mixture of papain and bromelain and a mixture of acetic acid and lactic acid. Amino acid composition analysis of collagen from comparative examples results are recorded in Table 6. Embodiments 1 through 3 had high protein concentrations of 1 mg/g to 1.56 mg/g, while Comparative Example 1 had a protein concentration of only 0.2 mg/g. In addition, proline, glycine, glutamine, and arginine are precursors of procollagen, and the higher content they are, the tighter and more concentrated the triple-helix structure of procollagen will be. As shown in Table 6, Embodiment 1 had a much higher amino acid content compared to Comparative Example 1.

TABLE 6

| | | Denaturation | | Protein | *Proline/Glycine/ |
|---|---|---|---|---|---|
| | | temperature | | concentration | Glutamine/Arginine |
| | Yield | (° C.) | Endotoxin (EU/ml) | (mg/g) | content (ppm) |
| Embodiment 1 | 55-80% | 88 | Non-gelling state <0.25 | 1.56 | 326/553/338/241 |
| Embodiment 2 | 45-60% | 77 | Non-gelling state <0.25 | 1.5 | — |
| Embodiment 3 | 30-45% | — | Non-gelling state <0.25 | 1 | — |
| Comparative Example 1 | 2-3% | 45 | Non-gelling state <0.25 | 0.2 | 127/374/75/58 |
| Comparative Example 2 | 0.5-1% | 45 | Non-gelling state <0.25 | | |

### ADVANTAGEOUS EFFECTS OF EMBODIMENTS

One of the advantageous effects of the extraction process described herein is that it replaces the existing technique of using NaOH cleaning while still effectively removing pigments and endotoxins from freshwater fish tissue to below 0.25 EU/ml, resulting in the extracted procollagen being clinically acceptable by medical standards.

Furthermore, the preparation time is shortened and yields of over 80% may be achieved. The preparation method of procollagen can increase the denaturation temperature of the extracted procollagen.

More procollagen can be extracted and retained, and the procollagen remains structurally intact with a tightly twisted triple-helix structure.

This process disclosed herein can be implemented in large-scale industrial production to produce procollagen that provides high value to the commercial and medical fields, promoting the development of biotechnology.

Therefore, the scope of the disclosure is defined not by the detailed description, but by the claims and their equivalents, and all variations within the scope of the claims and their equivalents are to be construed as being included in the disclosure.

What is claimed is:

1. A method of extracting procollagen comprising:
removing, by pressure extrusion, blood, water, and fat from freshwater fish tissue, wherein the removing does not include use of NaOH;
breaking down via air-blowing the extruded freshwater fish tissue into smaller freshwater fish tissue particles;
mixing the freshwater fish tissue particles with a nonionic surfactant, a salt electrolyte, and urea to obtain an emulsion solution;
homogenizing the emulsion solution for 13 minutes to 20 minutes;
refrigerating the emulsion solution at 0° C. to 10° C. for 5 minutes to 60 minutes;
washing the emulsion solution with water at 38° C. to 42° C. until an absorbance value of the emulsion solution at a monitored wavelength in a range of 200 nm to 300 nm approaches zero;
mixing the washed emulsion solution with an enzyme of papain, bromelain, or mixtures thereof and an acid solution of pH 3 to pH 6 to obtain an extract solution;
homogenizing the extract solution at 4° C. to 75° C. for 2 hours to 48 hours;
adding an enzyme inhibitor of acetic acid, lactic acid, or mixtures thereof to the extract solution to lower the pH to below 3;
homogenizing the inactivated extract solution at 4° C. to 75° C. for 2 hours to 48 hours; and
separating supernatant from the homogenized extract solution to obtain procollagen.

2. The method of extracting procollagen of claim 1, wherein the salt electrolyte is NaCl and the NaCl is added in an amount of between 1 wt % to 5 wt %.

3. The method of extracting procollagen of claim 1, wherein the urea is added in an amount of between 3 wt % to 10 wt %.

4. The method of extracting procollagen of claim 1, wherein the nonionic surfactant is selected from Tween 20, Tween 80, Triton X-100, or mixtures thereof, and added in an amount of between 0.05 wt % and 0.5 wt %.

5. The method of extracting procollagen of claim 1, wherein enzyme activity of the enzyme is between 20 and 2000 U, and the enzyme is added in an amount of between 0.5 wt % and 10 wt %.

6. The method of extracting procollagen of claim 1, wherein the acid solution is selected from a solution of acetic acid, citric acid, lactic acid, and mixtures thereof, and added in a concentration of between 0.5 M and 1 M.

7. The method of extracting procollagen of claim 1, wherein the enzyme is a combination of bromelain and papain with a ratio of bromelain to papain of between 0:1 and 1:2.

8. The method of extracting procollagen of claim 1, wherein the enzyme inhibitor is a mixture of acetic acid and lactic acid having a ratio of acetic acid to lactic acid of between 1:0 and 1:3.

9. The method of extracting procollagen of claim 1, wherein breaking down the freshwater fish tissue includes cutting and crushing the freshwater fish tissue to form powder, chunks, strips, and combinations thereof.

10. The method of extracting procollagen of claim 1, wherein the separating the supernatant from the homogenized extract solution includes filtrating, centrifuging, sieving, and combinations thereof.

11. The method of extracting procollagen of claim 1, wherein the removing and breaking down do not include an application of shear force on the fish tissue.
